SEQUENCE LISTING (16 sequences)

SEQ ID NO: 1 - protein, 51 aa, Glycine Max
MESQGQWNPLLSFSRFINHHSNHLATRLEETKRLAGTLIQSHTRTKPAFAA

SEQ ID NO: 2 - DNA, 246 bp, Aspergillus nidulans
cgggatagttccgacctaggattggatgcatgcggaaccgcacgagggcggggcggaaattgacacaccactcctctccacgcaccgttcaagaggtacgcgtatagagccgtatagagcagagacggagcactttctggtactgtccgcacgggatgtccgcacggagagccacaaacgagcggggccccgtacgtgctctcctaccccaggatcgcatccccgcatagctgaacatctatataa

SEQ ID NO: 3 - DNA, 33 bp, Artificial
  Transcription start Spacer
tacttagcccctccctcattgttaagggagcaa

SEQ ID NO: 4 - protein, 484 aa, Arabidopsis thaliana
MAAKKVFGSAEASNLVTELRRSFDDGVTRGYEWRVTQLKKLMIICDNHEPEIVAALRDDLGKPELESSVYEVSLLRNSIKLALKQLKNWMAPEKAKTSLTTFPASAEIVSEPLGVVLVISAWNYPFLLSIDPVIGAISAGNAVVLKPSELAPASSALLTKLLEQYLDPSAVRVVEGAVTETSALLEQKWDKIFYTGSSKIGRVIMAAAAKHLTPVVLELGGKSPVVVDSDTDLKVTVRRIIVGKWGCNNGQACVSPDYILTTKEYAPKLIDAMKLELEKFYGKNPIESKDMSRIVNSNHFDRLSKLLDEKEVSDKIVYGGEKDRENLKIAPTILLDVPLDSLIMSEEIFGPLLPILTLNNLEESFDVIRSRPKPLAAYLFTHNKKLKERFAATVSAGGIVVNDIAVHLALHTLPFGGVGESGMGAYHGKFSFDAFSHKKAVLYRSLFGDSAVRYPPYSRGKLRLLKALVDSNIFDLFKVLLGLA

SEQ ID NO: 5 - protein, 720 aa, Cannabis Sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 6 - protein, 385 aa, Cannabis Sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 7 - protein, 101 aa, Cannabis Sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 8 - protein, 398 aa, Cannabis Sativa
MGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 9 - protein, 515 aa, Cannabis Sativa
PRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 10 - DNA, 444 bp, Glycine Max
agccctttttgtatgtgctaccccacttttgtctttttggcaatagtgctagcaaccaataaataataataataataatgaataagaaaacaaaggctttagcttgccttttgttcactgtaaaataataatgtaagtactctctataatgagtcacgaaacttttgcgggaataaaaggagaaattccaatgagttttctgtcaaatcttcttttgtctctctctctctctcttttttttttttctttcttctgagcttcttgcaaaacaaaaggcaaacaataacgattggtccaatgatagttagcttgatcgatgatatctttaggaagtgttggcaggacaggacatgatgtagaagactaaaattgaaagtattgcagacccaatagttgaagattaactttaagaatgaagacgtcttatcaggttcttcatgactt

SEQ ID NO: 11 - DNA, 504 bp, Glycine Max
tgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaa

SEQ ID NO: 12 - DNA, 766 bp, Agrobacterium Tumefaciens
gtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatgcatattctatagtgtcacct

SEQ ID NO: 13 - DNA, 642 bp, Pissum Sativum
agctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaa

SEQ ID NO: 14 - protein, 545 aa, Cannabis Sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVDVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 15 - protein, 566 aa, Cannabis Sativa
MESQGQWNPLLSFSRFINHHSNHLATRLEETKRLAGTLIQSHTRTKPAFAAPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 16 - protein, 545 aa, Cannabis Sativa; identical to SEQ ID NO: 14

(19) United States
(12) Patent Application Publication
(10) Pub. No.: US 2024/0368613 A1
(43) Pub. Date: Nov. 7, 2024

(54) NON-ENDOGENOUS PRODUCTION OF CANNABINOIDS AND CANNABINOID PRECURSOR COMPOUNDS IN PLANT SYSTEMS

(71) Applicant: The Regents of the University of Colorado, a body Corporate, Denver, CO (US)

(72) Inventors: Brian S. DeDecker, Boulder, CO (US); Simon Kalmus, Boulder, CO (US)

(21) Appl. No.: 18/284,485

(22) PCT Filed: Apr. 13, 2022
(86) PCT No.: PCT/US22/24548

§ 371 Date: Sep. 27, 2023

Related U.S. Application Data
(60) Provisional application No. 63/174,106, filed on Apr. 13, 2021.

(51) Int. Cl. C12N 15/82; C12N 9/00; C12N 9/02; C12N 9/10; C12N 9/88

(52) U.S. Cl. CPC C12N 15/8243; C12N 9/0004; C12N 9/0008; C12N 9/1029; C12N 9/88; C12N 9/93; C12Y 102/01003; C12Y 121/03007; C12Y 121/03008; C12Y 203/01206; C12Y 404/01026

(57) ABSTRACT

The present invention includes novel systems, methods, and compositions for the non-endogenous production of cannabinoids in non-Cannabis plants, and preferably Glycine max or other high-biomass crops. In one embodiment, the present invention includes the generation of transgenic plant seeds, and preferably Glycine max seeds, expressing a heterologous nucleotide sequence encoding one or more heterologous enzymes necessary for the production a cannabinoid, a cannabinoid intermediate from a precursor compound. Certain precursors compound may be produced endogenously by the plant, or incorporated from a heterologous source and incorporated into the non-endogenous cannabinoid pathway.

### CROSS REFERENCE TO RELATED APPLICATIONS

This International PCT application claims the benefit of and priority to U.S. Provisional Application No. 63/174,106 filed Apr. 13, 2021, the specification, claims and drawings of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Apr. 11, 2022, is named “90245.00641_ST25” and is 41 Kbytes in size.

### FIELD OF INVENTION

The present invention relates to the heterologous production of therapeutically and commercially relevant compounds in transgenic plants. In particular, the present invention relates to novel systems, methods and compositions for the production of cannabinoid compounds and their precursors in transgenic plants, namely soybeans.

### BACKGROUND

A major difficulty in engineering plants to produce nonendogenous, (or heterologous) compounds such as cannabidiol (CBD), is the metabolic cost and developmental defects caused to the growing plant by the intermediates and products being produced. For instance, endogenous metabolites need to be shunted into the engineered non-endogenous pathway to produce CBD. Exogenous competition for essential metabolites such as malonyl-CoA for the production of compounds such as CBD typically cause growth and developmental defects in the transgenic plant. In addition, the nonendogenous compounds themselves may be toxic to the plant or potentially contaminate the local environment and water supply. Similar transgenic methods have been recently described for the production of cannabinoids in yeast (Luo et al., 2019). However, this application is also limited. For example, the method of using transgenic yeast requires the use of expensive bioreactors, intensive sterilization procedures, and remains limited by expensive separation and purification techniques. Moreover, there is significant clinical and commercial interest in minor phytocannabinoids from the Cannabis sativa plant. However, due to genetics it is difficult to produce appreciable quantities of these compounds due to competition for intermediates among specific cannabinoid pathways. As such, there is a long-felt need for an efficient and cost-effective system for the non-endogenous production of cannabinoid compounds.

### SUMMARY OF THE INVENTION

In one aspect, the present invention relates to the heterologous production of therapeutically and commercially relevant compounds in transgenic plants. In particular, the present invention relates to novel systems, methods and compositions for the production of cannabinoids, such as cannabidiol (CBD) in transgenic plants, namely soybeans. This method could also be used in seeds harvested from other commonly farmed crops such as corn, rice and rapeseeds. In another preferred embodiment, the present invention relates to novel systems, methods and compositions for the production of cannabinoid intermediates and precursors in transgenic plants, namely soybeans.

In another aspect, the present invention relates to the inducible heterologous production of therapeutically and commercially relevant compounds in transgenic plants. In particular, the present invention relates to novel systems, methods and compositions for the inducible production of cannabinoids, such as CBD in transgenic plants, namely soybeans seeds. In another embodiment, the present invention relates to novel systems, methods and compositions for the inducible production of cannabinoid intermediates and precursors and other compounds in transgenic plants, namely soybeans.

In another aspect, transgenic plant or seed of the invention may include a plant or seed, and preferably a soybean plant or seed, expressing a heterologous nucleotide sequence encoding one or more heterologous enzymes necessary for the production a cannabinoid, or a cannabinoid intermediate from a precursor compound, which may include one or more aldehydes. As noted above, the genes encoding the non-endogenous cannabinoid biosynthesis pathway may be subject to an inducible promoter, such as an AlcA/AlcR inducible expression system. The non-endogenous cannabinoid biosynthesis pathway may be encoded and expressed in a plant, and preferably a soybean plant, by a heterologous nucleotide sequence comprising one or more expression cassettes, operably linked to promoter(s), encoding one or more of the following heterologous enzymes: a heterologous Aldehyde Dehydrogenase enzyme, a heterologous Hexanoyl-CoA Synthetase enzyme, a heterologous Olivetolic Acid Synthase, a heterologous Olivetolic acid cyclase, a heterologous Prenyl Transferase, preferably including cannabigerolic acid (CBGA) synthase; and a heterologous CBDA synthase enzyme, or a heterologous THCA synthase enzyme.

Another embodiment if the invention includes the localized production of cannabinoid and cannabinoid intermediates from a non-endogenous cannabinoid biosynthesis pathway. In this embodiment, a cannabinoid synthase, such as CBDA synthase or the THCA synthase are coupled with localization signal forming a fusion peptide according to SEQ ID NO. 15 and 16, respectively, or a fragment or variant thereof. This localization signal may preferably be configured to direct cannabinoid biosynthesis in soybean plant, and may include a Translocon on the inner chloroplast envelope protein 22 TIC(22) localization signal, or a fragment or variant thereof.

In a preferred embodiment, a transgenic seed of the invention, whether directly transformed, or derived from a transgenic parent plant, may be contacted with a precursor such that the precursor is imbibed by the seed at levels greater than that of the wild-type level of said precursor. For example, in a transgenic seed of the invention may be contacted with an aldehyde precursor, and preferably hexanal, butyraldehyde or octanal. In this preferred embodiment, the exemplary precursor hexanal is incorporated into the non-endogenous cannabinoid pathway by the enzymatic action of aldehyde dehydrogenase forming hexanoic acid. The non-endogenous cannabinoid biosynthesis pathway is thus driven to produce one or more cannabinoid intermediates, Hexanoyl-CoA, 3,5,7-Trioxododecanoyl-CoA; and Olivetolic acid (OA), or a combination of the same. In another aspect, the non-endogenous cannabinoid pathway may use a non-endogenous aldehyde precursor, such as hexanal, butyraldehyde or octanal that may be incorporated into the cannabinoid biosynthesis pathway forming one or more of the following cannabinoids: cannabidiolic acid (CBDA); cannabidiol (CBD); tetrahydrocannabinolic acid (THCA); tetrahydrocannabinol (THC); cannabidivarinic acid (CBDVA); cannabidivarin (CBDV); cannabidiphorol (CBDP); tetrahydrocannabivarin (THCV); and/or tetrahydrocannabiphorol (THCP).

Additional aspects of the inventive technology will be evident from the detailed description and figures presented below.

### BRIEF DESCRIPTION OF THE FIGURES

FIG. 1: Method for production of compounds such as cannabinoids using transgenic plants. Transgenic plants are grown and harvested with no concurrent production of CBD. This eliminates the problem of developmental defects that would be caused to the plant by shunting endogenous metabolites to the production of exogenous compounds such as CBD. In addition, the local environment and water supply will not be potentially contaminated with compounds such as CBD. The harvested seeds can then be either stored for future production or transported immediately to a secure facility where they are imbibed and activated. Biosynthesis can be activated by one of two ways; either by addition of chemical pathway intermediates to the incubating the bean in a precursor compound, such as hexanal for the production of CBD and other cannabinoids, or by activating the expression of the transgenes by using an inducible system such as the AlcA/AlcR inducible expression system. After adequate incubation during which compound synthesis takes place, the biomass is processed. The compound of interest is then extracted from the processed biomass and refined.

FIG. 2: Design of the pathway for use of hexanal during seed germination for the biosynthesis of cannabidiol (CBDA). Using the method of inducing compound synthesis during seed germination as described (FIG. 1). Chemical precursors may be fed to the germinating seeds for incorporation into the nonendogenous compound synthesis pathway. As shown here, hexanal is fed to the germinating seeds to be incorporated into the CBD synthesis pathway. Feeding of the chemical precursor may also function as the inducer of the biosynthesis pathway during seed germination. A series of 6 transgenes of the CBD biosynthesis pathway then utilize the added hexanal and endogenous chemical intermediates of the plant to synthesize CBD. First, Aldehyde dehydrogenase (ALDH) is used to convert the hexanal to hexanoic acid. From there, Acyl-Activating Enzyme (AAE1) activates the hexanoic acid via Coenzyme A ligation, forming Hexanoyl-CoA. Next, the enzyme Olivetolic Acid Synthase (OLS) incorporates 3 endogenous Malonyl-CoA groups to form 3,5,7-Trioxododecanoyl-CoA, OLS then works in conjunction with OAC to cyclize the molecule forming olivetolic acid (OA). The enzyme Cannabigerolic Acid Synthase (CBGAS) prenylates OA with Geranyl Pyrophosphate (GPP) to form Cannabigerolic acid (CBGA). The CBGA is then converted into CBDA via the enzyme cannabidiolic acid synthase (CBDAS). Other precursors could be fed to the germinating seeds to make related compounds. For instance, butyraldehyde or octanal could be fed into the same pathway to produce CBDVA or CBDPA.

FIG. 3: Production of CBD in Soybean Tissue. Production of CBD in germinating soybean seeds of cultivar Williams 82. The seeds were infected with a DNA construct containing the CBD pathway genes shown in FIG. 2 via Agrobacterium strain EHA101. The chemical precursor hexanal was fed to the germinating seeds and the nonendogenous CBD pathway genes were induced with 0.4% EtOH via the AlcR/AlcA ethanol inducible expression system. The germinating seeds were incubated for 16 days on Gamborg B5 Medium. After the incubation, samples of the germinating seeds were homogenized via maceration. Organic compounds present in the sample were extracted with methanol and analyzed by triple quadrupole mass spectrometry. CBD production was detected in the germinating soybeans while other cannabinoids such as THC were not present.

FIG. 4: Control. Germinating soybean seeds were transfected as FIG. 3, but with a control DNA plasmid that does not contain the CBD pathway genes. The germinating seeds were fed hexanal and mock induced with ethanal as FIG. 3. Methanal extraction and analysis by triple quadrupole mass spectrometry showed no detectable CBD or any other cannabinoids.

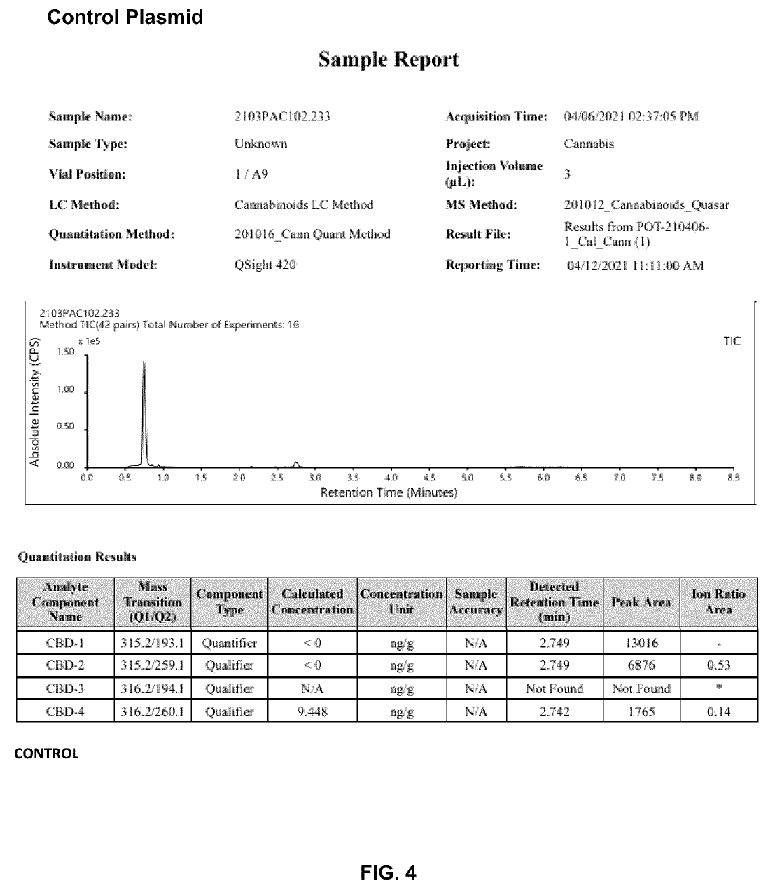

### DETAILED DESCRIPTION OF THE INVENTION

The present invention includes novel systems, methods, and compositions for the non-endogenous production of cannabinoids in a non-Cannabis plant. The present invention includes novel systems, methods, and compositions for the non-endogenous production of cannabinoids in a non-Cannabis plant, namely soybean seeds. An exemplary current transgenic plant would produce cannabinoids using soybeans that are amenable to modern farming practices and that allow for the more efficient and less expensive production of cannabinoids due to economies of scale. Soybean cultivation is much less expensive and more environmentally sustainable than the cultivation of Cannabis which is both labor and resource intensive.

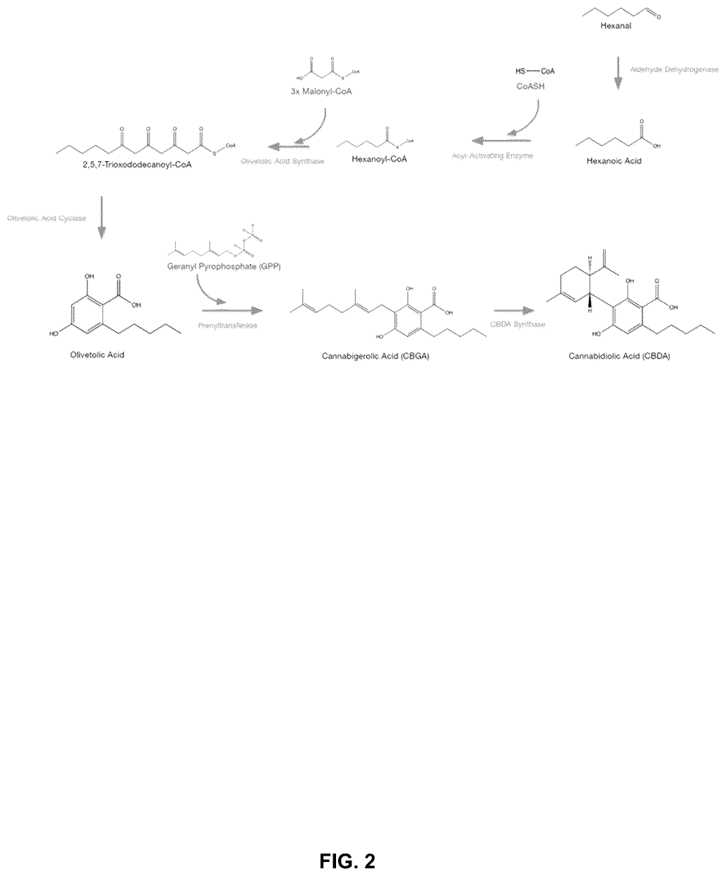

As noted above, traditional cultivation of CBD and other cannabinoids using the Cannabis plant is labor intensive to harvest and is often produced using indoor growing facilities that require intensive energy and land use. Extracting pure cannabinoids such as CBD and other minor cannabinoids from harvested Cannabis plants is also expensive. In comparison, the synthesized hydrophobic cannabinoids from the seeds of the transgenic plant are readily solubilized by the natural oil present in the soybean. The cannabinoid infused oils may then be easily extracted using current commercial methods for isolating oil from crop seeds. This is usually done by using expeller pressing or solvent-based extraction. Because the soybeans are incubated in a controlled environment, it is possible to add key intermediates for uptake by the soybean. For instance, hexanal was added to the germinating soybeans in this example to produce CBD. This can be applied for example to the biosynthesis other cannabinoids, cannabinoid intermediates and cannabinoid precursor compounds. For instance, in one example, transformed seeds could be butyraldehyde were added to the system, then the expected product of the same pathway described for CBDA (FIG. 2) would be CBDVA.

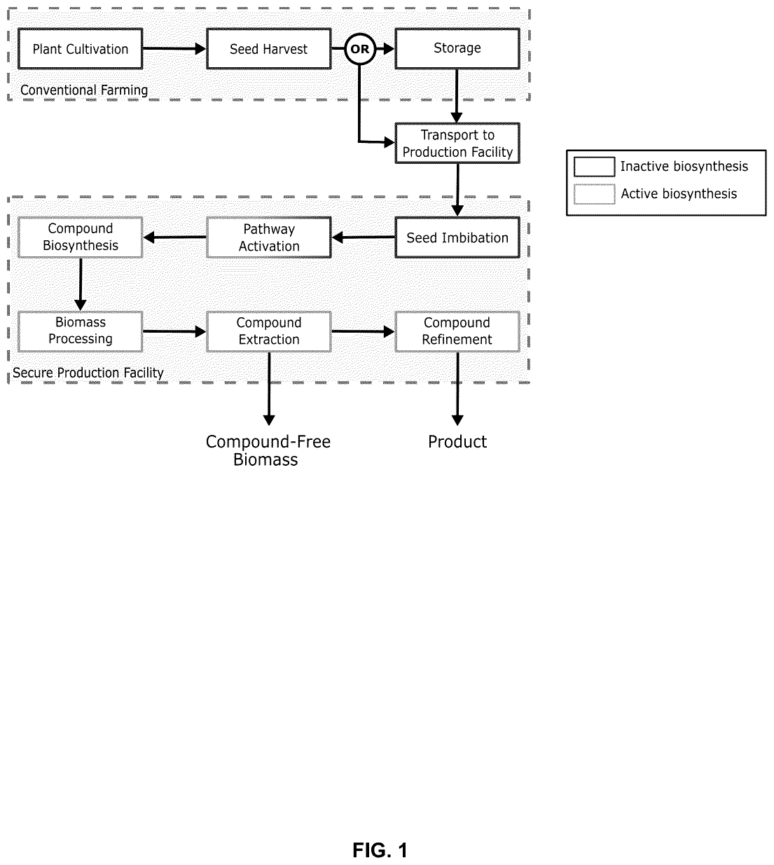

Another problem with traditional Cannabis plant cultivation is the presence of federally controlled substances such as tetrahydrocannabinol acid (THC) in the cannabis plants. In one embodiment, CBD produced in the transgenic soybean would be specific to the plant in regard to other cannabinoids. For instance, having no THCA synthase enzyme present, the transgenic plant would produce no THC. Naturally, the inverse could also be accomplished if THCA synthase were expressed in the transgenic seed producing THCA, and not CBDA. The present invention method mitigates these problems, as the compound is produced in seeds that have already been harvested from the fully matured plant (FIG. 1). The transgenic plant does not produce a non-endogenous cannabinoid, such as THC or CBD during development and growth of the plant in the field. The seeds of the transgenic plant are harvested and transported to a production facility. The seeds are germinated in a controlled environment and the synthesis of non-endogenous cannabinoid, such as THC or CBD is then initiated with an inducible system. This allows the engineered crop to not produce non-endogenous cannabinoid, such as THC or CBD, their intermediates or precursors, as well as the associated enzymes while growing in the field, thereby allowing for farming of the crop without developmental defects or potentially contaminating the local environment and water supply with synthesized compounds (FIG. 1).

Wild type (non-engineered) seeds may also be used in this method by transfecting them with the transgenic DNA after germination. For instance, the wild type seeds are harvested and germinated. Once germinated, the seeds are transfected with the transgenic DNA via the standard Agrobacterium mediated method. From there, the biosynthesis pathway is activated to produce compounds.

Known inducible promoter systems have been developed for plants such as the ethanol inducible expression system from Aspergillus nidulans (AlcR/AlcA). The Aspergillus nidulans AlcR/AlcA ethanol inducible expression system described in WO2001009357A2, by Syngenta Ltd., (incorporated herein by reference) can be used to induce expression of the synthetic metabolic pathway shortly after seed germination. Other examples of the method may utilize a similar inducible promoter system such as the estradiol dependent XVE system to control the timing of recombinant gene expression.

The biosynthesis pathway in the germinating beans may also be induced by adding an essential chemical pathway precursor to the system. The enzymes of the pathway are constitutively expressed, but the compound synthesis will not begin until a key chemical pathway precursor is added to the germinating seeds. For instance, all the enzymes of the CBDA pathway would be constitutively expressed except those needed to produce the key chemical precursor hexanal. Once hexanal is added to the germinating seeds, the biosynthesis pathway will then proceed and begin producing CBDA.

By controlling the genetic makeup of the plant used for production as well as the specific chemical precursors fed to the germinating seed, it is possible to produce, specifically the single desired minor cannabinoid. This eliminates substrate competition, by directing plant metabolism into production of a given minor cannabinoid. FIG. 2 displays the pathway that has been utilized in producing the cannabinoid CBD, and precursor for other cannabinoids such as CBD, in germinating soybean seeds. CBDV could be produced by this same nonendogenous pathway by feeding butyraldehyde instead of hexanal to the germinating seeds.

Additional embodiments may include utilizing the systems and methods of the invention for the non-endogenous production of therapeutically and commercially relevant compounds found in a variety of other plants. For example, hundreds of compounds called terpenes from the MEV and MEP pathways of plant metabolism are highly relevant to a vast array of modern industries, such as the cosmetic, pharmaceutical, and food industries, among others. However, they often exist in trace quantities or are difficult to extract due to limitations to cultivation or production. Additional embodiment may include utilizing the systems and methods of the invention for the nonendogenous production of other terpenes and alkaloids.

In another aspect, the present invention includes systems, methods and compositions to produce a transgenic plant having a non-endogenous cannabinoid biosynthesis pathway. In a preferred embodiment, the transgenic plant or seed of the invention may include a plant or seed, and preferably a soybean plant or seed, expressing a heterologous nucleotide sequence encoding one or more heterologous enzymes necessary for the production a cannabinoid, or a cannabinoid intermediate from a precursor compound, which may include one or more aldehydes. As noted above, the genes encoding the non-endogenous cannabinoid biosynthesis pathway may be subject to an inducible promoter, such as an AlcA/AlcR inducible expression system according to SEQ ID NO. 2, or a fragment or variant thereof.

In one preferred embodiment, the non-endogenous cannabinoid biosynthesis pathway may be encoded and expressed in a plant, and preferably a soybean plant, by a heterologous nucleotide sequence comprising an expression cassette, operably linked to a promoter, encoding one or more of the following heterologous enzymes: a heterologous Aldehyde Dehydrogenase enzyme, a heterologous Acyl-Activating enzyme, a heterologous Olivetolic Acid Synthase, a heterologous Olivetolic acid cyclase, a heterologous Prenyl Transferase comprising cannabigerolic acid (CBGA) synthase; and a heterologous CBDA synthase enzyme, or a heterologous THCA synthase enzyme., or, or fragments or variants of the same. In one preferred embodiment, the non-endogenous cannabinoid biosynthesis pathway may be encoded and expressed in a plant, and preferably a soybean plant, by a heterologous nucleotide sequence comprising an expression cassette, operably linked to a promoter, encoding one or more of the following heterologous enzymes: a heterologous amino acid sequence according to SEQ ID NO. 4, a heterologous amino acid sequence according to SEQ ID NO. 5, a heterologous amino acid sequence according to SEQ ID NO. 6, a heterologous amino acid sequence according to SEQ ID NO. 7, a heterologous amino acid sequence according to SEQ ID NO. 8; and a heterologous amino acid sequence according to ID NO. 9 or 14.

Another embodiment of the invention includes the localized production of cannabinoid and cannabinoid intermediates from a non-endogenous cannabinoid biosynthesis pathway. In this embodiment, a cannabinoid synthase, such as CBDA synthase or the THCA synthase are coupled with localization signal forming a fusion peptide. This localization signal may preferably be configured to direct cannabinoid biosynthesis in soybean plant, and may include a TIC22 localization signal according to SEQ ID NO. 22, or a fragment or variant thereof.

In another aspect, the present invention includes systems, methods and compositions for the non-endogenous production of cannabinoids and cannabinoid intermediates in a plant seed. In this embodiment, the invention may include a system for transforming a plant, and preferably a soybean plant to produce a transgenic seed expresses a heterologous nucleotide sequence, operably linked to a promoter, encoding one or more heterologous enzymes necessary for the production a cannabinoid, or a cannabinoid intermediate from a precursor compound. The non-endogenous pathway may be activated through contacting the transgenic seed with a quantity of a cannabinoid precursor compound, such as an aldehyde, which can be incorporated into the non-endogenous pathway.

In a preferred embodiment, a transgenic seed of the invention, whether directly transformed, or derived from a transgenic parent plant, may be contacted with a precursor such that the precursor is imbibed by the seed at levels greater than that of the wild-type level of said precursor. For example, in a transgenic seed of the invention may be contacted with an aldehyde precursor, and preferably hexanal or butyraldehyde. In this preferred embodiment, the exemplary precursor hexanal is incorporated into the non-endogenous cannabinoid pathway by the enzymatic action of aldehyde dehydrogenase forming hexanoic acid. The non-endogenous cannabinoid biosynthesis pathway is thus driven to produce one or more cannabinoid intermediates, Hexanoyl-CoA, 3,5,7-Trioxododecanoyl-CoA; and Olivetolic acid (OA).

The OA produced by the non-endogenous cannabinoid biosynthesis pathway may be converted into a cannabinoid intermediate by a prenyl transferase, in this case CBGA synthase (SEQ ID NO. 8) to form CBGA. The CBGA produced non-endogenous cannabinoid biosynthesis pathway of the invention may further be converted into one or more cannabinoids through the enzymatic action of a cannabinoid synthase. For example, a heterologous CBDA synthase enzyme according to amino acid sequence SEQ ID NO. 9, or a heterologous THCA synthase enzyme according to amino acid sequence SEQ ID NO. 14, or a fragment or variant thereof.

In another aspect, the present invention includes systems, methods and compositions for the non-endogenous production of cannabinoids and cannabinoid intermediates. In this preferred aspect, the invention may include the step of transforming a non-Cannabis plant, and preferably a soybean plant or other high-biomass crop/plant, to express a heterologous nucleotide sequence, operably linked to a promoter, encoding one or more heterologous enzymes necessary for the production a cannabinoid, or a cannabinoid intermediate from a precursor compound. Expression of the non-endogenous cannabinoid biosynthesis pathway may be induced, for example by inducing the promoter, or contacting a precursor to the plant system such that the precursor is incorporated into the non-endogenous pathway. The resulting cannabinoids, and cannabinoid intermediate may be isolated from the plant biomass and further purified for commercial, therapeutic, or recreational use. In a preferred embodiment, the non-endogenously produced cannabinoid of the invention may include cannabidiolic acid (CBDA), cannabidiol (CBD), tetrahydrocannabinolic acid, cannabidivarinic acid (CBDVA), or cannabidivarin (CBDV), cannabidiphorolic acid (CBDPA), cannabidiphorol (CBDP), tetrahydrocannabinolic acid (THCA), tetrahydrocannabinol (THC), tetrahydrocannabivarin acid (THCVA), tetrahydrocannabivarin (THCV), tetrahydrocannabiphorolic acid (THCPA), tetrahydrocannabiphorol (THCP) and a cannabinoid intermediate of the invention may include cannabigerolic acid (CBGA) or cannabigerol (CBG).

In another aspect, the present invention includes systems, methods and compositions for the non-endogenous production of precursors for cannabinoids and cannabinoid intermediates. In this preferred embodiment, transgenic plant may be configured to express a heterologous nucleotide sequence, operably linked to a promoter, encoding one or more heterologous enzymes necessary for the production a one or more of cannabinoid precursors, which may include: Hexanoic acid, Hexanoyl-CoA, 3,5,7-Trioxododecanoyl-CoA; and Olivetolic acid (OA), or a combination of the same.

Additional aspects of the invention include isolated nucleotide sequences and expression vectors encoding one or more enzymes necessary to non-endogenously produce a cannabinoid, cannabinoid intermediate, or cannabinoid precursor compounds, or a combination of the same. For example, in one preferred embodiment, the invention may include an expression vector having a nucleotide sequence, operably linked to a promoter, encoding one or more of the following: a heterologous Aldehyde Dehydrogenase enzyme; a heterologous Acyl-Activating enzyme; a heterologous Olivetolic Acid Synthase; a heterologous Olivetolic acid cyclase; a heterologous Prenyl Transferase comprising cannabigerolic acid (CBGA) synthase; and a heterologous CBDA synthase enzyme, or a heterologous THCA synthase enzyme, all of which may be part of a single or separate expression cassette.

A plant or seed may be transformed by one of the expression vectors of the invention producing one or more cannabinoid precursors, while optionally producing a cannabinoid intermediate or cannabinoid. In this embodiment, the cannabinoid precursors may be isolated from the biomass, or may be used as feedstock for a separate cannabinoid production system, such as an in vitro production system (PCT/IB2015/056445), a cannabinoid production bioreactor (PCT/IB2015/056445, incorporated herein by reference), a yeast-based cannabinoid production system (PCT/US2018/029668, incorporated herein by reference), or a bacterial-based production system (PCT/US2018/054400, incorporated herein by reference), where they may be further converted into a cannabinoid intermediate or cannabinoid.

As used herein, a “cannabinoid” is a chemical compound (such as cannabinol, THC or cannabidiol) that is found in the plant species Cannabis among others like: Echinacea; Acmella Oleracea; Helichrysum umbraculigerum; Radula marginata (Liverwort) and Theobroma cacao, and metabolites and synthetic analogues thereof that may or may not have psychoactive properties. Cannabinoids therefore include (without limitation) compounds (such as THC) that have high affinity for the cannabinoid receptor (for example Ki<250 nM), and compounds that do not have significant affinity for the cannabinoid receptor (such as cannabidiol, CBD). Cannabinoids also include compounds that have a characteristic dibenzopyran ring structure (of the type seen in THC) and cannabinoids which do not possess a pyran ring (such as cannabidiol). Hence a partial list of cannabinoids includes THC, CBD, dimethyl heptylpentyl cannabidiol (DMHP-CBD), 6,12-dihydro-6-hydroxy-cannabidiol (described in U.S. Pat. No. 5,227,537, incorporated by reference); (3S,4R)-7-hydroxy-46-tetrahydrocannabinol homologs and derivatives described in U.S. Pat. No. 4,876,276, incorporated by reference; (+)-4-[4-DMH-2,6-diacetoxy-phenyl]-2-carboxy-6,6-dimethylbicyclo[3.1.1]hept-2-en, and other 4-phenylpinene derivatives disclosed in U.S. Pat. No. 5,434,295, which is incorporated by reference; and cannabidiol (−)(CBD) analogs such as (−)CBD-monomethylether, (−)CBD dimethyl ether; (−)CBD diacetate; (−)3′-acetyl-CBD monoacetate; and ±AF11, all of which are disclosed in Consroe et al., J. Clin. Phannacol. 21:428S-436S, 1981, which is also incorporated by reference. Many other cannabinoids are similarly disclosed in Agurell et al., Pharmacol. Rev. 38:31-43, 1986, which is also incorporated by reference.

Examples of cannabinoids are tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C4, cannabidivarinic acid, cannabidiorcol, delta-9-tetrahydrocannabinolic acid A, delta-9-tetrahydrocannabinolic acid B, delta-9-tetrahydrocannabinolic acid-C4, delta-9-tetrahydrocannabivarinic acid, delta-9-tetrahydrocannabivarin, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol,trihydroxy-delta-9-tetrahydrocannabinol, and cannabinol. Examples of cannabinoids within the context of this disclosure include tetrahydrocannabinol and cannabidiol. The term “cannabinoid” may also include different modified forms of a cannabinoid such as a hydroxylated cannabinoid or cannabinoid carboxylic acid. For example, if a UGT were to be capable of glycosylating a cannabinoid, it would include the term cannabinoid as defined elsewhere, as well as the aforementioned modified forms. It may further include multiple glycosylation moieties.

As used herein, an “intermediate cannabinoid” comprises a cannabinoid that can be further converted into another cannabinoid. For example, CBGA, as an intermediate cannabinoid, can be converted into THCA or CBDA. Notably, identification of the acidic form of a cannabinoid, also explicitly includes the decarboxylated form as described here. As used herein, a “precursor” includes all chemical compounds along the biosynthetic pathway that that precede a cannabinoid or cannabinoid intermediate.

As used herein, the term “aldehyde” means a hydrocarbon having the formula RCHO characterized by an unsaturated carbonyl group (C═O). In a preferred embodiment, the aldehyde is any aldehyde made from a fatty acid or fatty acid derivative. In one embodiment, the R group is at least about 1, 2, 3, 4, 5, 6, 7, 8, 9, 10, 11, 12, 13, 14, 15, 16, 17, 18, 19, or 20 carbons in length.

A polypeptide can be expressed in monocot plants and/or dicot plants. Techniques for introducing nucleic acids into plants are known in the art, and include, without limitation, Agrobacterium-mediated transformation, viral vector-mediated transformation, electroporation, and particle gun transformation (also referred to as biolistic transformation). See, for example, U.S. Pat. No. 5,538,880; U.S. Pat. No. 5,204,253; U.S. Pat. No. 6,329,571; and U.S. Pat. No. 6,013,863; Richards et al., Plant Cell. Rep. 20:48-20 54 (2001); Somleva et al., Crop Sci. 42:2080-2087 (2002); Sinagawa-Garcia et al., Plant Mol Biol (2009) 70:487-498; and Lutz et al., Plant Physiol., 2007, Vol. 145, pp. 1201-1210. In some instances, intergenic transformation of plastids can be used as a method of introducing a polynucleotide into a plant cell. In some instances, the method of introduction of a polynucleotide into a plant comprises chloroplast transformation. In some instances, the leaves and/or stems can be the target tissue of the introduced polynucleotide. If a cell or cultured tissue is used as the recipient tissue for transformation, plants can be regenerated from transformed cultures if desired, by techniques known to those skilled in the art.

Other suitable methods for introduce polynucleotides include electroporation of protoplasts, polyethylene glycol-mediated delivery of naked DNA into plant protoplasts, direct gene transformation through imbibition (e.g., introducing a polynucleotide to a dehydrated plant), transformation into protoplasts (which can comprise transferring a polynucleotide through osmotic or electric shocks), chemical transformation (which can comprise the use of a polybrene-spermidine composition), microinjection, pollen-tube pathway transformation (which can comprise delivery of a polynucleotide to the plant ovule), transformation via liposomes, shoot apex method of transformation (which can comprise introduction of a polynucleotide into the shoot and regeneration of the shoot), sonication-assisted Agrobacterium transformation (SAAT) method of transformation, infiltration (which can comprise a floral dip, or injection by syringe into a particular part of the plant (e.g., leaf)), silicon-carbide mediated transformation (SCMT) (which can comprise the addition of silicon carbide fibers to plant tissue and the polynucleotide of interest), electroporation, and electrophoresis. Such expression may be from transient or stable transformations.

The term “homolog” or “variant,” used with respect to an original enzyme or gene of a first family or species, refers to distinct enzymes or genes of a second family or species which are determined by functional, structural or genomic analyses to be an enzyme or gene of the second family or species which corresponds to the original enzyme or gene of the first family or species. Most often, homologs or variant will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes. A “fragment” used with respect to an original enzyme or gene refers to a truncated portion of the peptide or gene that still retains its intended function.

The term “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. An “inducible” promoter may be a promoter which may be under environmental control. Tissue-specific, tissue-preferred, cell type specific, and inducible promoters constitute the class of “non-constitutive” promoters. A “constitutive” promoter is a promoter which may be active under most environmental conditions or in most cell or tissue types.

As used herein, the term “transformation” or “genetically modified” refers to the transfer of one or more nucleic acid molecule(s) into a cell. A plant is “transformed” or “genetically modified” by a nucleic acid molecule transduced into the plant when the nucleic acid molecule becomes stably replicated by the plant. As used herein, the term “transformation” or “genetically modified” encompasses all techniques by which a nucleic acid molecule can be introduced into, such as a plant.

The term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; or can be regulatory in nature, etc. There are various types of vectors including virus, plasmid, bacteriophages, cosmids, and bacteria. An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of a cassette assists in the assembly of the expression vectors. An expression vector is a replicon, such as plasmid, phage, virus, chimeric virus, or cosmid, and which contains the desired polynucleotide sequence operably linked to the expression control sequence(s).

As is known in the art, different organisms preferentially utilize different codons for generating polypeptides. Such “codon usage” preferences may be used in the design of nucleic acid molecules encoding the proteins and chimeras of the invention in order to optimize expression in a particular host cell system. For example, all nucleotides of the present invention may be optimized for expression in a select organisms such a Glycine max.

A polynucleotide sequence is operably linked to an expression control sequence(s) (e.g., a promoter and, optionally, an enhancer) when the expression control sequence controls and regulates the transcription and/or translation of that polynucleotide sequence.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. The Table below, contains information about which nucleic acid codons encode which amino acids.

### Amino Acid Nucleic Acid Codons

| Amino Acid | Nucleic Acid Codons |
|---|---|
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |

Moreover, because the proteins are described herein, one can chemically synthesize a polynucleotide which encodes these polypeptides/chimeric proteins. Oligonucleotides and polynucleotides that are not commercially available can be chemically synthesized e.g., according to the solid phase phosphoramidite triester method first described by Beaucage and Caruthers, Tetrahedron Letts. 22:1859-1862 (1981), or using an automated synthesizer, as described in Van Devanter et al., Nucleic Acids Res. 12:6159-6168 (1984). Other methods for synthesizing oligonucleotides and polynucleotides are known in the art. Purification of oligonucleotides is by either native acrylamide gel electrophoresis or by anion-exchange HPLC as described in Pearson & Reanier, J. Chrom. 255:137-149 (1983).

The term “plant” or “plant system” includes whole plants, plant organs, progeny of whole plants or plant organs, embryos, somatic embryos, embryo-like structures, protocorms, protocorm-like bodies (PLBs), and culture and/or suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like). The invention may also include Glycine max plants or seeds.

The term “expression,” as used herein, or “expression of a coding sequence” (for example, a gene or a transgene) refer to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “nucleic acid” or “nucleic acid molecules” include single-and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), mRNA (messenger RNA), miRNA (micro-RNA), hpRNA (hairpin RNA), tRNA (transfer RNA), whether charged or discharged with a corresponding acetylated amino acid), and cRNA (complementary RNA). The term “deoxyribonucleic acid” (DNA) is inclusive of cDNA, genomic DNA, and DNA-RNA hybrids. The terms “nucleotide sequence” and “nucleotide sequence segment,” or more generally “sequence,” will be understood by those in the art as a functional term that includes both genomic sequences, ribosomal RNA sequences, transfer RNA sequences, messenger RNA sequences, operon sequences, and smaller engineered nucleotide sequences that encoded or may be adapted to encode, peptides, polypeptides, or proteins.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide. It should be noted that any reference to a SEQ ID, or sequence specifically encompasses that sequence, as well as all corresponding sequences that correspond to that first sequence. For example, for any amino acid sequence identified, the specific specifically includes all compatible nucleotide (DNA and RNA) sequences that give rise to that amino acid sequence or protein, and vice versa.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hair-pinned, circular, and padlocked conformations. The term “sequence identity” or “identity,” as used herein in the context of two nucleic acid or polypeptide sequences, refers to the residues in the two sequences that are the same when aligned for maximum correspondence over a specified comparison window.

The terms “approximately” and “about” refer to a quantity, level, value, or amount that varies by as much as 30%, or in another embodiment by as much as 20%, and in a third embodiment by as much as 10% to a reference quantity, level, value or amount. As used herein, the singular form “a,” “an,” and “the” include plural references unless the context clearly dictates otherwise.

As used herein, “heterologous” or “exogenous” in reference to a nucleic acid is a nucleic acid that originates from a foreign species, or is synthetically designed, or, if from the same species, is substantially modified from its native form in composition and/or genomic locus by deliberate human intervention. A heterologous protein may originate from a foreign species or, if from the same species, is substantially modified from its original form by deliberate human intervention. By “host cell” is meant a cell which contains an introduced nucleic acid construct and supports the replication and/or expression of the construct.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1: Production of Cannabidiol CBD Through Spatiotemporal Control of Recombinant Genes

The present inventors have introduced transgenes into soybean seeds (Glycine max) that are able to synthesize the small molecule compound cannabidiol (CBD) during germination. The compound can be traditionally found in the Cannabis sativa plant, while a transgenic soybean plant could produce large quantities of CBD for production purposes. This process is less expensive than labor-intensive and resource-heavy cannabis production and allows for the production of transgenic soybeans that are free of THC.

In one embodiment the invention includes a nucleotide expression construct that has been configured to inducible express the enzymes needed to synthesize the compound CBD in plants. Testing by mass spec has demonstrated that the construct successfully produces CBD in tissue samples from soybean. The method was shown to produce 400 picograms per gram of germinating seed. As shown in FIG. 2, an exemplary nonendogenous pathway for production of CBD of the inventions includes:

Aldehyde Dehydrogenase (ALDH): Hexanal was added to the headspace of the germinating soybean seeds. Once diffused into the seed, nonendogenous aldehyde dehydrogenase oxidizes the hexanal to form hexanoic acid. The aldehyde dehydrogenase from Arabidopsis thelania ALDH3H1 (SEQ ID NO. 4) was used as an exemplary aldehyde dehydrogenase to oxidize the fed hexanal into hexanoic acid.

Acyl-Activating Enzyme (AAE1): The next step is the addition of the endogenous compound Coenzyme A (CoA) to hexanoic acid to form Hexonyl-CoA. The Acyl-Activating Enzyme from Cannabis sativa AAE1 (SEQ ID NO. 5) is used for this addition reaction.

Olivetolic Acid Synthase (OLS): The next step is the sequential addition of three molecules of endogenous Malonyl-CoA to the Hexanoyl-CoA intermediate to form the unstable 3,5,7-Trioxododecanoyl-CoA intermediate. The type III polyketide synthase Cannabis sativa OLS (SEQ ID NO. 6) is used for this addition reaction.

Olivetolic acid cyclase (OAC): The next step is to cyclize the unstable 3,5,7-Trioxododecanoyl-CoA intermediate to form the final product of cannabidiol (CBD). The DABB protein Cannabis sativa CBDC (SEQ ID NO. 7) is used for this cyclization reaction.

Prenyl Transferase (CBGAS): The next step is the claisen condensation of the endogenously produced geranyl pyrophosphate (GPP) with the olivetolic acid from the previous step. The product of this reaction if cannabigerolic acid (CBGA) (SEQ ID NO. 8)

CBDA Synthase: The final step is the production of cannabidiolic acid from the CBGA intermediate via Cannabidiolic Acid Synthase. (NCBI Accession number A6P6V9) The synthase was fused with the (TIC22) localization signal (SEQ ID NO. 9) on the N-terminus to target the enzyme to the soluble fraction of the chloroplast envelope.

THCA Synthase: The final step is the production of THCA from the CBGA intermediate via THCA Synthase. (SEQ ID NO. 14) The synthase may be fused with the TIC22 localization signal (SEQ ID NO. 1) on the N-terminus to target the enzyme to the soluble fraction of the chloroplast envelope.

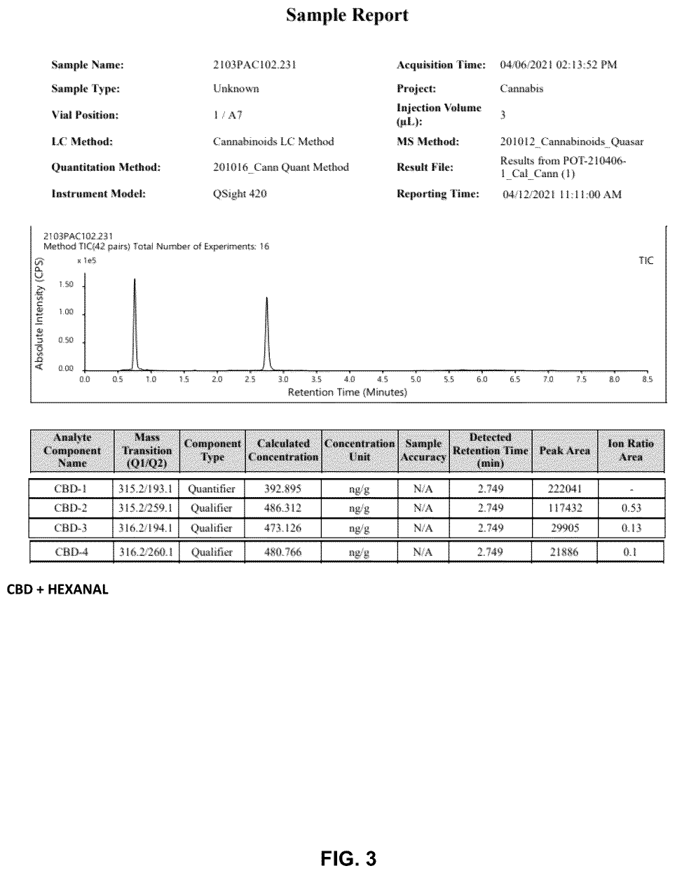

Oxidation of CBDA and THCA: Hexanal is known to induce the production of reactive oxygen species in the chloroplast and this environment likely decarboxylated the CBDA and THCA products forming CBD and THC respectively (FIG. 3) during the incubation of the germinating soybean seed. Plasmids were constructed using a modified Golden Braid technique in order to assemble the DNA sequences synthesized by Twist Biosystems before insertion into a modified Agrobacterium binary plasmid.

All heterologous pathway protein coding sequences were driven the inducible AlcA/AlcR system with the AlcA promoter sequence according to SEQ ID NO. 2. A 33 base pair spacer (SEQ

ID NO. 3) was utilized between the transcription start site and translation start site for all 6 exogenous cannabinoid pathway enzymes (SEQ ID NO. 4-9) shown in FIG. 2. Terminators according to SEQ ID NO. 10-13 were used to comprise the 3′ untranslated regions (3′ UTR) of the transgenes.

1-42. (canceled)

43. A system of non-endogenous cannabinoid production comprising:
establishing a seed of a non-Cannabis plant that expresses a heterologous nucleotide sequence, operably linked to a promoter, encoding one or more heterologous enzymes necessary for the production a cannabinoid, or a cannabinoid intermediate from a precursor compound;
contacting a quantity of precursor compound with said seed inducing production of a cannabinoid, or a cannabinoid intermediate compounds.

44. The system of claim 43 wherein said precursor compound comprises an aldehyde.

45. The system of claim 44 wherein said aldehyde is contacted with said transgenic plant in non-endogenous levels compared to wild-type level of said aldehyde.

46. The system of claim 44 wherein said aldehyde comprises hexanal, butyraldehyde or octanal.

47. The system of claim 43 wherein said plant comprises a soybean plant.

48. The system of claim 47 wherein said soybean plant comprises a soybean seed.

49. The system of claim 43 wherein said promoter comprises an inducible promoter.

50. The system of claim 49 wherein said inducible promoter comprises an AlcA/AlcR inducible expression system.

51. The system of claim 50 wherein said AlcA/AlcR inducible expression system comprises the nucleotide sequence according to SEQ ID NO. 2.

52. The system of claim 43 wherein the heterologous nucleotide sequence comprises an expression cassette, operably linked to a promoter, encoding one or more of the following heterologous enzymes:
a heterologous Aldehyde Dehydrogenase enzyme, or a fragment or variant thereof;
a heterologous Acyl-Activating enzyme, or a fragment or variant thereof;
a heterologous Olivetolic Acid Synthase, or a fragment or variant thereof;
a heterologous Olivetolic Acid cyclase, or a fragment or variant thereof;
a heterologous Prenyl Transferase comprising cannabigerolic acid (CBGA) synthase, or a fragment or variant thereof; and
a heterologous CBDA synthase enzyme, or a heterologous THCA synthase enzyme, or a fragment or variant thereof.

53. The system of claim 52 wherein the CBDA synthase or the THCA synthase are coupled with a localization signal forming a fusion peptide according to SEQ ID NO. 15 and 16, respectively, or a fragment or variant thereof.

54. The system of claim 53 wherein said localization signal comprises a TIC22 localization signal.

55. The system of claim 43 wherein the heterologous nucleotide sequence comprises an expression cassette, operably linked to a promoter, encoding the following heterologous enzymes:
a heterologous amino acid sequence according to SEQ ID NO. 4, or a fragment or variant thereof;
a heterologous amino acid sequence according to SEQ ID NO. 5, or a fragment or variant thereof;
a heterologous amino acid sequence according to SEQ ID NO. 6, or a fragment or variant thereof;
a heterologous amino acid sequence according to SEQ ID NO. 7, or a fragment or variant thereof;
a heterologous amino acid sequence according to SEQ ID NO. 8, or a fragment or variant thereof; and
at least one of a heterologous amino acid sequence according to SEQ ID NO. 9 or 14, or a fragment or variant thereof.

56. The system of claim 43 wherein at least one of a heterologous amino acid sequence according to SEQ ID NO. 9 or 14 include a localization signal forming a fusion peptide according to SEQ ID NO. 15 and 16, respectively, or a fragment or variant thereof.

57. The system of claim 56 wherein said localization signal comprises a localization signal according to SEQ ID NO. 1.

58. The system of claim 43 wherein said cannabinoid precursor comprises one or more of the following:
Hexanoic acid;
Hexanoyl-CoA;
3,5,7-Trioxododecanoyl-CoA; and
Olivetolic acid (OA).

59. The system of claim 43 wherein said cannabinoid intermediate comprises cannabigerolic acid (CBGA), or Cannabigerol (CBG).

60. The system of claim 43 wherein said cannabinoid comprises one or more of the following:
cannabidiolic acid (CBDA);
cannabidiol (CBD);
tetrahydrocannabinolic acid (THCA);
tetrahydrocannabinol (THC);
cannabidivarinic acid (CBDVA);
cannabidivarin (CBDV);
cannabidiphorol (CBDP);
tetrahydrocannabivarin (THCV); or
tetrahydrocannabiphorol (THCP).

61. The system of claim 43 wherein said seed comprises a seed directly transformed by Agrobacterium mediated transformation.

62. The system of claim 43 wherein said seed comprises a seed derived from a plant transformed by Agrobacterium mediated transformation.

63-115. (canceled)
